(19) United States
(12) Patent Application Publication
(10) Pub. No.: US 2006/0280778 A1
(43) Pub. Date: Dec. 14, 2006

(54) Homeopathic Teething Pain Relief Composition and Method

(76) Inventor: Rachel Wilson, San Clemente, CA (US)

Correspondence Address: QUICKPATENTS, INC., 31877 DEL OBISPO, SUITE 202, SAN JUAN CAPISTRANO, CA, 92675, US

(21) Appl. No.: 11/307,459

(22) Filed: Feb. 8, 2006

Related U.S. Application Data
(60) Provisional application No. 60/688,049, filed on Jun. 8, 2005.

(51) Int. Cl. A61K 47/00

(52) U.S. Cl. 424/439

(57) ABSTRACT

A composition for the relief of symptoms of teething in human children, and a method of its use, is disclosed. The composition includes effective amounts of the homeopathic ingredients Belladonna 3C, Chamomilla 3C and Coffea Cruda 3C, preferably combined with distilled water to form a solution. The solution is introduced into a flexible tube that is open at one end and sealed at another end. The open end is then sealed to form a water-tight pouch, which is then cooled such that the solution is frozen. In use, a pouch may be opened and the solution expelled partially from the tube in order to be orally administered to the gums of the child. The volume of solution in each pouch contains enough of the components of the solution to be effective at providing pain relief to the child experiencing teething pain, as the solution melts, while the cold temperature of the solution provides further pain relief for the child. A sweetener, artificial and natural flavors, and artificial colors may be included in the solution to enhance its taste and appearance.

### PRIORITY CLAIM

This application claims the benefit of U.S. Provisional Application 60/688,049, filed on Jun. 8, 2005.

### STATEMENT REGARDING FEDERALLY SPONSORED RESEARCH AND DEVELOPMENT

Not Applicable

### FIELD OF THE INVENTION

This invention relates to homeopathic remedies and, more specifically, to homeopathic remedies reducing pain and discomfort in teething children.

### DISCUSSION OF RELATED ART

Various topical compositions for treating teething pain in children have been devised, many of which contain an analgesic or anesthetic, such as acetaminophen or benzocaine. However, many parents do not wish to introduce such medicines to their children, and prefer more natural, homeopathic remedies. Homeopathic remedies for teething pain, however, are difficult to administer with any lasting effect. One such product, sold under the brand name Camilia® by Boiron, is a liquid that is administered orally and contains Belladonna 5C, Chamomilla 9C, and Ferrum phosphoricum 5C, all of which are products produced by, for example, Laboratoires Boiron in France. Camilia provides some momentary relief, yet quickly dissipates. Indeed, doses are recommended every 15 minutes.

Perhaps the most common teething pain remedy over the last several decades is a chilled gel-filled teether device, such as that disclosed in U.S. Pat. No. 5,782,868 to Moore, Jr. et al on Jul. 21, 1998. Such a device is chilled in a refrigerator or freezer and then given to the child to chew on. The cold and pliable gel-filled ring provides comfort to the child while allowing the child to satisfy his chewing instincts while teething. However, such a device quickly attains room temperature, and the cold soothing effect is quickly lost with such a device. To be useful again, the device must be placed back into the refrigerator or freezer for a period of time, during which the child is without remedy. Further, such a device does not make any provision for dispensing a pain relief composition, and thus this device relies on purely mechanical means for relieving pain.

Other devices, such as those taught in U.S. Pat. No. 5,908,440 to McCloskey et al. on Jun. 1, 1999, and U.S. Pat. No. 6,863,681 to Dickerson on Mar. 8, 2005, teach teething devices that can dispense either pain relieving medicines or compositions or foodstuffs. Such device, while perhaps effective at dispensing such compositions, make no provision for lowering the temperature of the child's gums to provide cooling relief to gum soreness or irritation. Further, such devices must be refilled frequently, which can be inconvenient to parents. Such devices are further ill-suited at holding a liquid homeopathic remedy, since such devices are not water-tight.

Topically applied gels or creams for relieving teething pain are usually administered at room temperature and quickly dissipate. Such gels and creams (such as those sold under the brand-name Orajel®, for example) are effective at relieving pain quickly, but the pain relief is short-lived. Further, such topically applied compositions, whether homeopathic or otherwise, do little to help a child fulfill his instinctual desires to chew, which helps teeth break-through gums during teething and helps relieve pain associated with teething.

Therefore, there is clearly a need for a homeopathic, effective composition for relieving teething pain that also allows a child to chew. Such a needed composition would further provide for cooling relief for teething pain, as well as being in a convenient package that is easily transported, stored, and used. Such a needed composition would be effective for a considerable duration, and would provide lasting relief to the child. The present invention accomplishes these objectives.

### SUMMARY OF THE INVENTION

The present invention is a composition for the relief of symptoms of teething in human children. The composition includes effective amounts of the homeopathic ingredients Belladonna 3C, Chamomilla 3C, and Coffea Cruda 3C, all of which are products produced by, for example, Laboratoires Boiron in France under Homeopathic Pharmacopoeia of the United States (HPUS) regulations and guidelines. Preferably these ingredients are further diluted with distilled water to form a solution. The solution is introduced into a flexible tube that is open at one end and sealed at another end. The open end is then sealed to form a water-tight pouch. Such pouches are purchased by a consumer, who places each pouch into a freezer for freezing the solution. When needed, a pouch may be removed from the freezer, opened, and then expelled partially from the tube to be orally administered to the gums of the child. The volume of solution in each pouch contains enough of the components of the solution to be effective at providing pain relief to the child experiencing teething pain, as the solution melts, while the cold temperature of the solution provides further pain relief for the child.

Preferably the solution also includes an effective amount of a sweetener for masking the taste of the Belladonna 3C, Chamomilla 3C and Coffea Cruda 3C. Such a sweetener, for example, may be high-fructose corn syrup. Still further, the solution may include citric acid, other natural flavors, and artificial flavors to further aid in masking the taste of the active ingredients.

A preservative such as potassium sorbate may be included, as well, such that the pouches filled with the solution may be preserved while being transported, stored, and displayed in a retail environment, for example.

The present invention is a homeopathic, effective composition for relieving teething pain. The present composition also allows a child to fulfill the instinctual cravings to chew while teething. Further, the present composition provides for cooling relief for teething pain, as well as being in a convenient package that is easily transported, stored, and used. The present composition is effective for a considerable duration, providing lasting relief to the child. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the invention, illustrating a frozen solution of the invention partially expelled from a flexible tube of the invention, as applied to the gums of a human;

FIG. 2 is a top plan view of the invention, illustrating the solution in the flexible tube sealed at both ends thereof;

FIG. 3 is a side elevational view of the invention;

FIG. 4 is a front elevational view of the invention; and

FIG. 5 is a side elevational view of the invention, illustrating a series of water-tight pouches of the invention.

### DETAILED DESCRIPTION OF THE PREFERRED EMBODIMENT

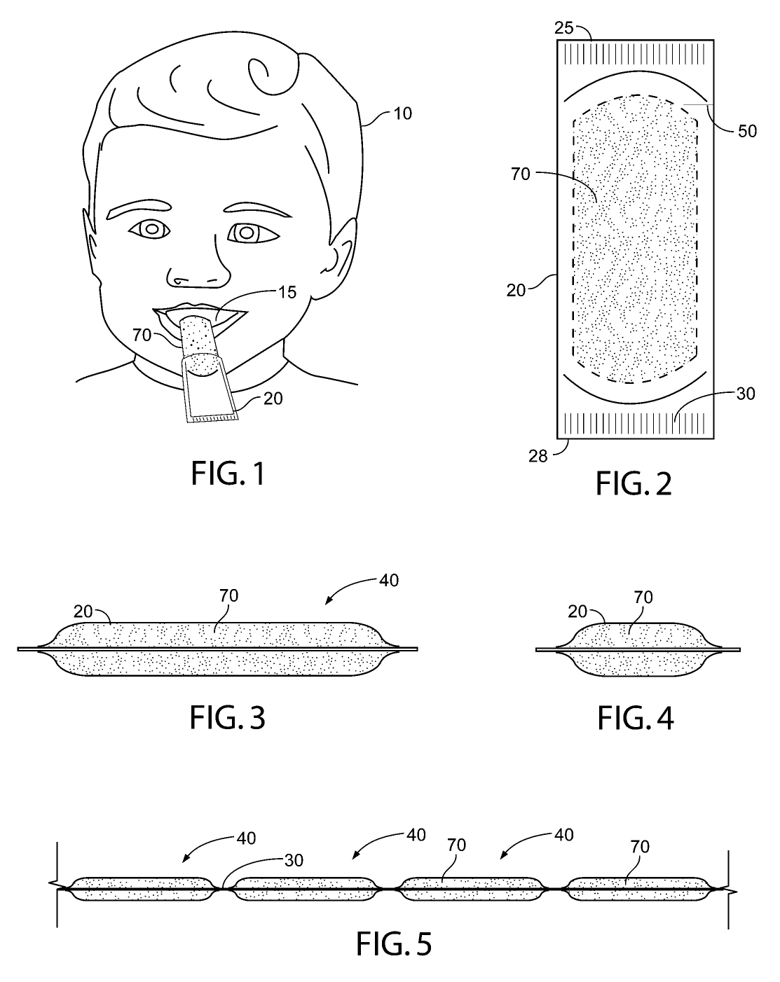

The present invention is a composition for the relief of symptoms of teething in humans 10, such as infants and children (FIG. 1). The composition includes effective amounts of the homeopathic ingredients Belladonna 3C, Chamomilla 3C and Coffea Cruda 3C, preferably combined with distilled water to form a solution 70.

The solution 70 is introduced into a flexible tube 20, such as polyurethane, santoprene, flexible vinyl (FPVC), low density and linear low density polyethylene (LDPE and LLDPE), flexible nylon, or the like, that is open at one end 25 and sealed at another end 28 (FIG. 2). The open end 25 is then sealed, such as by forming a heat seal 30 across the tube 20, to form a water-tight pouch 40 (FIGS. 3-5). For efficient production, such water-tight pouches 40 may be made in a string along the length of the flexible tube 20 (FIG. 5), and then cut into individual pouches 40. At this stage the pouches 40 may be sold along supply chains until they are ultimately purchased by a consumer, who places each pouch 40 into a freezer (not shown) for freezing the solution 70.

When needed, a pouch 40 with the frozen solution 70 may be removed from the freezer and cut open at one end 25. An optional score line 50 may be included in one side of each pouch 40 such that the pouch 40 may be opened manually without the need to cut the pouch 40 open with scissors, a knife, or the like (FIG. 5).

The frozen solution 70 is then manually expelled partially from the tube 20 and orally administered to the gums 15 of the human child 10, such as illustrated in FIG. 1. The volume of solution 70 in each pouch 40 contains enough of the components of the solution 70 to be effective at providing pain relief to the child 10 experiencing teething pain, as the solution 70 melts, while the cold temperature of the solution provides further pain relief for the child 10.

Preferably the solution 70 also includes an effective amount of a sweetener for masking the taste of the Belladonna 3C, Chamomilla 3C and Coffea Cruda 3C. Such a sweetener, for example, may be high-fructose corn syrup. Still further, the solution 70 may include citric acid, other natural flavors, and artificial flavors to further aid in masking the taste of the active ingredients. Artificial colors may also be introduced, such that the solution 70 appears to the child 10 to be more a frozen popsicle than a homeopathic pain-relief remedy.

A preservative such as potassium sorbate may be included, as well, such that the pouches 40 filled with the solution 70 may be preserved while being transported, stored, and displayed in a retail environment, for example. Importantly, however, all ingredients must combine to form a solution 70 with a freezing point no lower than 30 degrees Fahrenheit such that the solution 70 will freeze in a standard consumer freezer.

While greater amounts of the ingredients of Belladonna 3C, Chamomilla 3C, and Coffea Cruda 3C, when combined, can be used in the solution 70, it has been found that about 5% to 10% by weight of the combination of these ingredients is effective. There appears to be a synergistic effect when these three ingredients are combined in approximately equal amounts, although each ingredient may range from 1% to 5% for the solution 70 and still show some effect.

Additionally, other homeopathic ingredients may be included, such as Aconitum Napellus, Calcarea Phosphorica, Hyperioum, and Magnesia Phosphorica. These ingredients have been found to be less effective than Belladonna 3C, Chamomilla 3C, and Coffea Cruda 3C, but still may show some effect. Even with such additional ingredients, however, preferably the combination of these with Belladonna 3C, Chamomilla 3C, and Coffea Cruda 3C comprise no more than 10% to 15% of the solution 70, by weight, and preferably these optional ingredients combined constitute no more than 5% of the solution 70 by weight.

Further, between 15% and 20% of high-fructose corn syrup, by weight, effectively masks the taste of the other active ingredients. The balance of the solution 70 is comprised of the other natural and artificial flavors and colors. All of the ingredients are combined with preferably about 0.5 oz. distilled water to form the solution 70. Such a volume of solution 70 results in a pouch 40 size of approximately 1 inch by 3.3 inches, which is a suitable size for the child 10 to grasp and manipulate easily.

While a particular form of the invention has been illustrated and described, it will be apparent that various modifications can be made without departing from the spirit and scope of the invention. For example, other homeopathic or other ingredients may be added. Accordingly, it is not intended that the invention be limited, except as by the appended claims.

What is claimed is:

1. A composition for the relief of the symptoms of teething in infants and children, comprising:
a. an effective amount of Belladonna 3C;
b. an effective amount of Chamomilla 3C; and
c. an effective amount of Coffea Cruda 3C.

2. The composition of claim 1 in distilled water to form a solution.

3. The composition of claim 2 further including an effective amount of a sweetener for masking the taste of the Belladonna 3C, Chamomilla 3C and Coffea Cruda 3C.

4. The composition of claim 2 further including citric acid.

5. The composition of claim 2 further including natural and artificial flavors.

6. The composition of claim 2 further including potassium sorbate as a preservative.

7. The composition of claim 2 further including a water-tight flexible tube for containing the solution.

8. The composition of claim 7 wherein the solution is frozen.

9. A method of treating the symptoms of teething in a human comprising the steps of:
a. freezing an effective dose of the solution of claim 2;
b. orally administering said solution to the gums of the human until the solution is substantially melted.

10. A method of treating the symptoms of teething in a human comprising the steps of:
a. providing a flexible tube open at one end;
b. introducing an effective dose of the solution of claim 2 into the flexible tube;
c. sealing the open end of the flexible tube to form a water-tight pouch;
d. freezing the solution inside the flexible tube;
e. opening one end of the flexible tube to expose the frozen solution;
f. orally administering the solution to the gums of the human until the solution is substantially melted.
